(19) United States
(12) Patent Application Publication
(10) Pub. No.: US 2022/0028484 A1
(43) Pub. Date: Jan. 27, 2022

(54) Multiplex PCR Kit for identifying human genotype profile using new combination of mini STRs and method for identifying human genotype profile using the same

(71) Applicant: Republic of Korea (National Forensic Service Director Ministry of Interior and Safety), Wonju-si (KR)

(72) Inventors: Eung Soo KIM, Gunpo-si (KR); Youn Hyoung NAM, Pyeongtaek-si (KR); Jeongyong KIM, Wonju-si (KR)

(21) Appl. No.: 17/367,498

(22) Filed: Jul. 5, 2021

(30) Foreign Application Priority Data
Jul. 21, 2020 (KR) 10-2020-0090456

(51) Int. Cl. G16B 20/20; C12Q 1/686

(52) U.S. Cl. CPC G16B 20/20; C12Q 1/686

(57) ABSTRACT

The present invention provides a multiplex PCR kit with improved STR analysis success rate by introducing mini-STRs at 7 gene loci including a mini-STR at SE33 locus, which has high gene identification sensitivity, into a human genotype profile identification technique, as well as a method for identification of human genotype profiles using the same. According to the present invention, the STR analysis success rate is improved by identifying an STR at a locus that cannot be discriminated depending on DNA degradation level, thereby offering a lot of DNA information. Further, the present invention may contribute to more efficient DNA identification such as extension of database record, increase in identity determination success rate, etc. Still further, clues for reconstruction of a crime scene may be offered while improving reliability of forensic investigation results, simultaneously, thereby contributing to solving the crime.

### TECHNICAL FIELD

The present invention relates to a multiplex PCR kit for identifying a human genotype profile using a new mini-STR combination, as well as a method for identifying a human genotype profile using the same. More particularly, the present invention relates to a multiplex PCR kit with improved STR analysis success rate by introducing mini-STRs at 7 gene loci including mini-STR at SE33 locus, which has high gene identification sensitivity, into a human genotype profile identification technique, as well as a method for identification of human genotype profiles using the same.

Further, the present invention relates to a technology enabling gene identification even if genomic DNA in forensic specimens collected on a crime scene was degraded or denatured, including: introducing mini-STRs at 7 gene loci including mini-STR at SE33 locus, which has high gene identification sensitivity, into a human genotype profile identification technique, so that reliability of results of scientific (or forensic) investigation may be increased and, at the same time, clues for reconstruction of the crime scene may be offered, thereby contributing to solving crimes.

### BACKGROUND ART

Forensic science supporting criminal investigations has rapidly developed due to rapid development of scientific and engineering knowledge and analysis methods. For scientific (or forensic) investigations, physical analysis, chemical analysis, molecular genetic analysis and multidisciplinary scientific analysis are performed on forensic science specimens, which are various samples collected at crime scenes or large disaster sites. The results obtained from such analysis have a key role in proving a crime or revealing the cause of an accident with regard to the forensic investigation. In particular, detection and analysis of human genetic DNA in forensic specimens collected at a crime scene may offer primary clues to determining identity of incident people (that is, victim and suspect) and reconstruction of a crime scene, thereby contributing to solving the crime.

Analysis of human genome DNA in the forensic specimen collected at the crime scene, that is, genotype profile identification (abbrev. to “gene identification”) is generally used in forensic investigations. Genomic DNA of a victim or suspect can be extracted from different pieces of evidence and may be analyzed to analogize the existence of the suspect at the crime scene and/or who inflicted bodily injury on the victim. In general, in the forensic application, gene identification is implemented by amplifying specific DNA sites in chromosomes (STR regions in autosomes and sex chromosomes) based on PCR and analyzing the genotype of STR alleles.

An STR (short tandem repeat) existing at a specific locus of the chromosome depends on and varies between individuals. Therefore, allele genotypes are analyzed by determining the number of repeats of STRs at specific loci, thereby enabling gene identification for determining identity of incident people. For example, with regard to an STR at a specific locus, if a person has 12 repetitions and an allele genotype repeating 19 times at the paternal side while having 15 repetitions and an allele genotype repeating 18 times at the maternal side, a first descendent of the person may have 12 repetitions as well as 15 repetitions or 18 repetitions, and further have 19 repetitions as well as 15 repetitions or 18 repetitions.

On the basis of CODIS (combined DNA index system) core STR loci known at present, for example, CODIS 20, amelogenin, D18S51, FGA, D21S11, D8S1179, vWA, D13S317, D16S539, D7S820, TH01, D3S1358, D5S818, CSF1P0, D2S1338, D19S433, D1S1656, D12S391, D2S441, D10S1248, DYS391, etc., gene identification is implemented and, according to the gene identification for further analysis of loci with high discrimination (e.g., SE33, Penta E, Penta D) including CODIS 20, the identity of an incident person is discovered. Therefore, it is necessary to analyze all possible loci in forensic specimens such as field examination products or samples of incident people so as to success DNA examination.

However, DNA extracted from samples collected at an incident scene is often exposed for a long period to contamination due to external substances, strong heat, ultraviolet (UV) light, etc. and, sometimes, may not remain intact. In such a case, DNA in the extracted forensic specimen may be degraded or extracted in a smaller size than desired due to biochemical reaction or oxidation. As such, in the case where a DNA sample is degraded or denatured, when STR analysis is conducted through existing capillary electrophoresis (CE), STR allele genotype analysis results cannot be obtained at a locus where an amplified product has a large size. Therefore, as a result of DNA examination, STR allele genotype analysis at a locus where the amplified product has a large size fails and, instead, allele genotype analysis result may be obtained only at a locus where some amplified product has a small size. Therefore, it often occurs that the result is inappropriate for inclusion in a related database or identity determination is impossible.

Currently, GlobalFiler™ PCR amplification card (manufactured by ThermoFisher Scientific Co.) and PowerPlex® fusion system (manufactured by Promega Co.), which are commercially available and broadly used in STR analysis for the purpose of forensic examination, have entailed such a limitation that some forensic specimens cannot be subjected to gene identification. When a DNA sample is degraded or denatured, variable regions (HV1, HV2) of mitochondrial DNA (mtDNA) may be analyzed. However, due to consumption of a long time and maternal inheritance of mitochondria, there is a limit in application of the above analysis method to all subjects to be examined.

As a result of intensively repeating studies to develop STR analysis techniques that enable analysis of even degraded or denatured DNA samples and exhibit further improved gene identification sensitivity, the present inventors have developed a novel gene identification technique using a new mini-STR combination, capable of detecting DNA samples that could not be analyzed using the existing GlobalFiler™ PCR amplification card (manufactured by ThermoFisher Scientific Co.) and PowerPlex® fusion system (manufactured by Promega Co.).

Meanwhile, the subject matters described in the background art are provided only for promoting understanding of the background of the present invention, and are not recited as recognition that these can be used as “prior art” of the present invention.

### PRIOR ART DOCUMENT

[Patent Document]

(Patent Document 1) Korean Registered Patent Publication No. 10-1533792 (Jul. 6, 2015)

(Patent Document 2) Korean Registered Patent Publication No. 10-1902508 (Oct. 2, 2018)

### DISCLOSURE

### Technical Problem

It is an object of the present disclosure to provide a multiplex PCR kit for identifying a human genotype profile using a new mini-STR combination, as well as a method for identifying a human genotype profile using the same. More particularly, the present invention relates to a multiplex PCR kit with improved STR analysis success rate by introducing mini-STRs at 7 gene loci including mini-STR at SE33 locus, which has high gene identification sensitivity, into a human genotype profile identification technique, as well as a method for identification of a human genotype profile using the same.

Further, another object of the present invention is to provide technology enabling gene identification even if genomic DNA in forensic specimens collected at a crime scene was degraded or denatured, including: introducing mini-STRs at 7 gene loci including a mini-STR at SE33 locus, which has high gene identification sensitivity, into a human genotype profile identification technique, so that reliability of results of scientific (or forensic) investigation may be increased and, at the same time, clues for reconstruction of the crime scene may be offered, thereby contributing to solving the crime.

However, the subject of the present invention as described above is exemplary, and the scope of the present invention is not limited thereby. Further, other objects and advantages of the present invention will become more apparent from the detailed description, claims and accompanying drawings of the present invention.

### Technical Solution

As a result of repeating studies to solve the above technical problems and to meet the objects of the present invention described above, the present inventors have proposed a multiplex PCR kit with improved STR analysis success rate by introducing mini-STRs at 7 gene loci including a mini-STR at SE33 locus, which has high gene identification sensitivity, into a human genotype profile identification technique, as well as a method for identification of a human genotype profile using the same.

In other words, the inventors have developed a multiplex PCR kit for human genotype profile identification using a new mini-STR combination by: selecting a total of 7 loci at which an amplified product of the STR region has a large size of 300 bp or more (including SE22 locus having high gene identification sensitivity), among STR (short tandem repeat) loci; and applying the same to the mini-STR analysis technique. Further, the multiplex PCR kit of the present invention is further provided with a pair of primers that specifically amplifies one sex discrimination locus, that is, the amelogenin locus.

In particular, the multiplex PCR kit used for human genotype profile identification using mini-STR combination according to the present invention may be configured to analyze a mini-STR at SE33 locus and a mini-STR at TPOX locus, which were impossible to be analyzed by means of mini-STR multiplex PCT kits commercially available for similar purposes such as AmpFLSTR™Minifiler™ PCR amplification kit (manufactured by ThermoFisher Scientific co.), thereby improving gene identification sensitivity.

The term “sample” or “forensic science specimen” as used herein refers to various samples collected at crime scenes or large-scale disaster sites, and means a sample subjected to physical analysis, chemical analysis, molecular genetic analysis, and various other scientific analyses for forensic investigation. Forensic specimens may include various evidence at crime scenes, from which genes are detected, for example, saliva, blood, bloodstains, semen, vaginal fluid, vaginal secretions, vaginal wall discharges (e.g., vaginal secretions collected using a specimen collection tool such as a cotton swab, vaginal epithelium) or mixtures thereof, hair, body hair, sweat, dead skin cells or mixtures thereof, clothes, underwear, handkerchiefs, masks, cigarette butts, cups, beverage bottles, cans, tissue paper, or the like. However, the forensic specimen of the present invention is not limited to the above substances and various samples collected at a crime scene, from which human genomic DNA could be detected, may of course be used in the present invention.

The term “gene sample” as used herein is used in a broad sense including DNA, RNA, cDNA generated from RNA by reverse transcriptase, DNA or cDNA amplified through pre-PCR (preparatory PCR), or the like.

The term “STR (short tandem repeat) allele” as used herein refers to a gene having a specific repeat number of STR, which is a repeat unit of a specific base sequence useable for individual discrimination. For example, when the repeat number of STR allele is 16, the allele may be indicated by allele 16.

The term “mini-STR” as used herein may mean a technique itself that reduces a size of amplified product including STR region by altering or changing a design of the primer (e.g., shifting a position of the primer) in which the STR region at a specific locus is specifically amplified; and/or a small STR region (an STR region or fragment having a shorter length than the STR region to be amplified by the primer before design change) as a subject to be amplified for gene identification using such a technique as described above. Further, the term “multiplex PCR” means simultaneously analyzing a plurality of STR allele types through PCR amplification of multiple loci in a single reactor.

The present invention provides a multiplex PCR kit used for human genotype profile identification (genotype analysis) using a combination of mini-STRs (“a mini-STR combination”).

The multiplex PCR kit used for human genotype profile identification (“gene identification”) using a mini-STR combination according to one embodiment of the present invention may include: a pair of primers of SEQ ID NOS: 1 and 2, which specifically amplifies STR region at CSF1P0 locus to produce a mini-STR amplification product at CSF1P0 locus; a pair of primers of SEQ ID NOS: 3 and 4, which specifically amplifies STR region at D7S820 locus to produce a mini-STR amplification product at D7S820 locus; a pair of primers of SEQ ID NOS: 5 and 6, which specifically amplifies STR region at TPOX locus to produce a mini-STR amplification product at TPOX locus; a pair of primers of SEQ ID NOS: 7 and 8, which specifically amplifies STR region at FGA locus to produce a mini-STR amplification product at FGA locus; a pair of primers of SEQ ID NOS: 11 and 12, which specifically amplifies STR region at D2S1338 locus to produce a mini-STR amplification product at D2S1338 locus; a pair of primers of SEQ ID NOS: 13 and 14, which specifically amplifies STR region at D13S317 locus to produce a mini-STR amplification product at D17S317 locus; and a pair of primers of SEQ ID NOS: 15 and 16, which specifically amplifies STR region at SE33 locus to produce a mini-STR amplification product at SE33 locus.

The multiplex PCR kit used for gene identification using a mini-STR combination according to one embodiment of the present invention may further include a pair of primers of SEQ ID NOS: 9 and 10, which specifically amplifies the amelogenin locus for sex discrimination.

The multiplex PCR kit used for gene identification using a mini-STR combination according to one embodiment of the present invention may further include DNA polymerases, dNTPs, PCR buffer and a marker of PCR amplification product.

With regard to the multiplex PCR kit used for gene identification using a mini-STR combination according to one embodiment of the present invention, a first marker may be labeled at both of the 5′ end of the primer of SEQ ID NO: 1 and the 5′ end of the primer of SEQ ID NO: 3, a second marker may be labeled at both of the 5′ end of the primer of SEQ ID NO: 5 and the 5′ end of the primer of SEQ ID NO: 7, a third marker may be labeled at the 5′ end of the primer of SEQ ID NO: 9, the 5′ end of the primer of SEQ ID NO: 11 and the 5′ end of the primer of SEQ ID NO: 13, and a fourth marker may be labeled at the 5′ end of the primer of SEQ ID NO: 15.

With regard to the multiplex PCR kit used for gene identification using a mini-STR combination according to one embodiment of the present invention, the above first marker, second marker, third marker and fourth marker may be fluorescent materials detected at different wavelengths, respectively.

Accordingly, the multiplex PCR kit used for gene identification using a mini-STR combination according to one embodiment of the present invention may discriminate or distinguish mini-STR amplification products labeled with such fluorescent materials detected at different wavelengths, respectively, through spectroscopic techniques, thereby enabling multiplex PCR analysis that simultaneously analyzes a plurality of STR allele types in only one examination.

A method for identification of a human genotype profile using a mini-STR combination according to one embodiment of the present invention may include: preparing a gene sample from a specimen; amplifying the gene sample through multiplex PCR using the multiplex PCR kit that is used for human genotype profile identification using a mini-STR combination described above; and determining STR allele types corresponding to CSF1P0 locus, D7S820 locus, TPOX locus, FGA locus, D2S1338 locus, D13S317 locus and SE33 locus, respectively, based on the result of the multiplex PCR.

The method for identification of a human genotype profile using a mini-STR combination according to one embodiment of the present invention may distinguish a male or female gender by XX type or XY type analysis of the amelogenin locus.

Further, the method for identification of a human genotype profile using a mini-STR combination according to one embodiment of the present invention may discriminate the mini-STR amplification products labeled with fluorescent materials detected at different wavelengths, respectively, through spectroscopic techniques, thereby enabling multiplex PCR analysis that simultaneously analyzes a plurality of STR allele types by only one examination.

Meanwhile, the 5′ end of a forward primer may be labeled with any marker such as Cy5, Cy3, x-rhodamine, Texas red, SYBR green, 6-FAM, FAM, VIC, JOE, NED, HEX, TET, PET and TAMRA.

### Advantageous Effects

The multiplex PCR kit used for human genotype profile identification using a new mini-STR combination according to the present invention may discriminate mini-STRs at some loci that could not be discriminated on the basis of DNA degradation by GlobalFiler™ PCR amplification kit and PowerPlex® fusion system commercially available in the art, so as to further attain discrimination sensitivity, which is a shortcoming of the prior art. Further, there is an advantage of improving precision in identity determination by discriminating a mini-STR at SE locus with high gene identification sensitivity. Accordingly, the present invention may discriminate mini-STRs at unidentified loci based on DNA degradation so as to improve an STR analysis success rate, thereby offering a lot of DNA information. Moreover, the present invention may contribute to more efficient DNA identification such as extension of database records, increase in identity determination success rate, etc.

Further, the present invention may enable gene identification using the multiplex PCR kit used for human genotype profile identification using a new mini-STR combination even if human genomic DNA in a forensic specimen collected at a crime scene was degraded or denatured, whereby reliability of forensic investigation results may be increased while offering clues for reconstruction of the crime scene, thereby contributing to solving the crime.

However, the scope of the present invention is not limited to the aforementioned effects.

### DESCRIPTION OF DRAWINGS

FIG. 1 is a schematic view illustrating a size distribution of a multiplex PCP amplification product when amplified by the multiplex PCR kit used for human genotype profile identification using a mini-STR combination according to the present invention.

FIG. 2 is a schematic view illustrating comparison of the mini-STR multiplex PCT kit of the present invention with GlobalFiler™ PC R amplification kit and PowerPlex® fusion system with regard to the size distribution of a multiplex PCR amplification product.

FIG. 3 is a flowchart illustrating a process of human genotype profile identification (STR allele type analysis) using the mini-STR multiplex PCR kit of the present invention.

FIG. 4 shows a graph of STR analysis result of 2800M standard DNA using the mini-STR multiplex PCR kit of the present invention, wherein STR allele type at each locus is discriminated.

FIGS. 5A and 5B show graphs of STR analysis results using the conventional kits, that is, GlobalFiler™ PCR amplification kit and PowerPlex® fusion system, respectively, wherein STR allele types at some loci could not be discriminated.

FIG. 5C shows a graph of STR analysis results using the mini-STR multiplex PCR kit of the present invention, wherein the STR allele types not discriminated by the conventional STR analysis PCR amplification kits as illustrated in FIGS. 5A and 5B could be discriminated.

### DETAILED DESCRIPTION OF PREFERRED EMBODIMENTS OF INVENTION

Hereinafter, the present invention will be described in more detail through the following examples. However, the following examples are merely illustrative of the content of the present invention and do not limit the scope of the present invention. What can be easily inferred by those skilled in the art from the detailed description and examples of the present invention is construed as being within the scope of the present invention. References cited in the present invention are incorporated herein by reference.

Throughout the specification, when a part “includes” a certain component, this means that other components may be further included rather than excluding other components unless specifically stated to the contrary.

### Example 1: Design and Preparation of Primer Net

To fabricate the mini-STR multiplex PCT kit of the present invention, seven (7) loci (e.g., SE33, CSF1P0, D7S820, D2S1338, TPOX and FGA), at which large-scale amplified products contained in the GlobalFiler™ PCR amplification kit and the PowerPlex® fusion system that are used for individual identification in the National Forensic Investigation Research, were selected, and STR regions at these total 7 loci were specifically amplified into amplified products in a small size, followed by preparing a primer set capable of analyzing STR allele types at corresponding loci, respectively. Further, a pair of primers capable of specifically amplifying the amelogenin locus for sex determination was also prepared. Information of the prepared primer set is shown in Table 1 below (each color in Table 1 indicates color of emitted light labeled at the 5′ end of each forward primer).

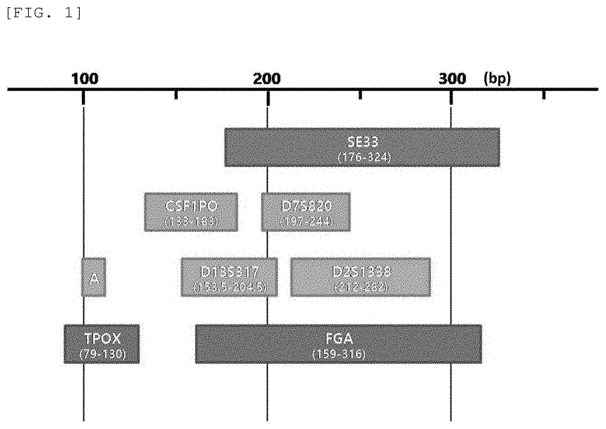

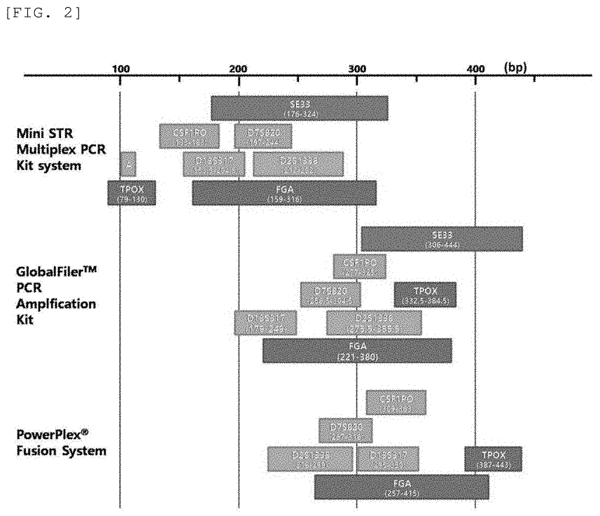

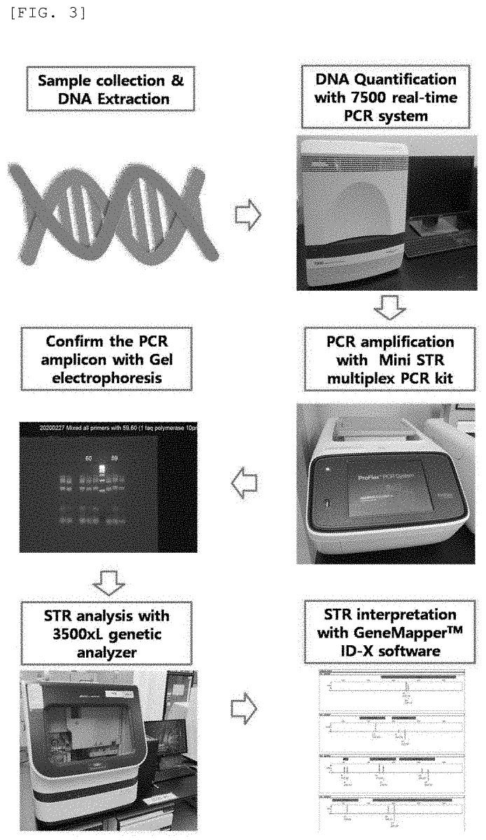

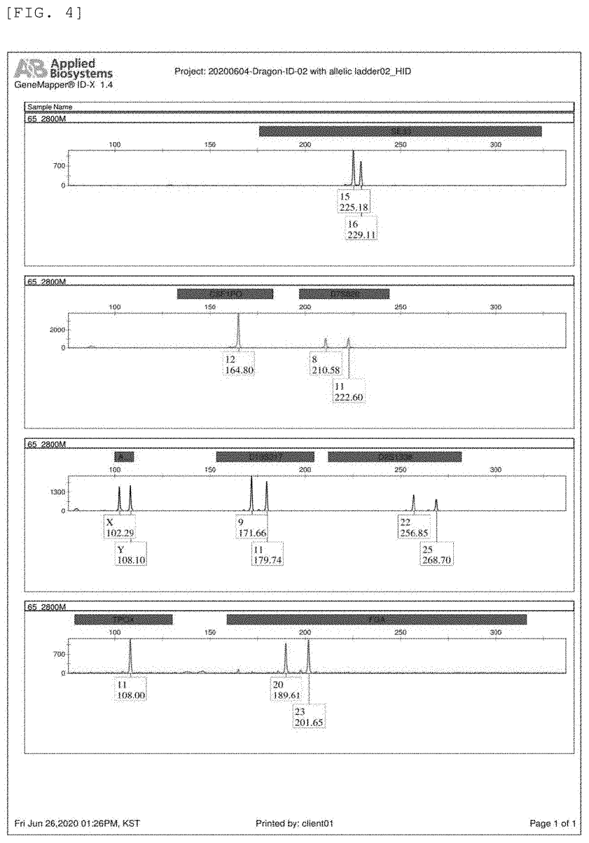

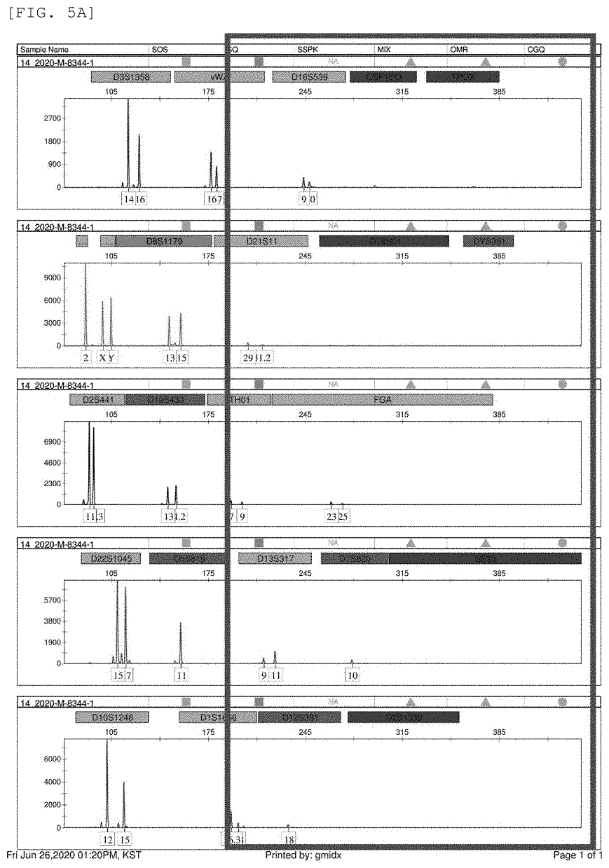

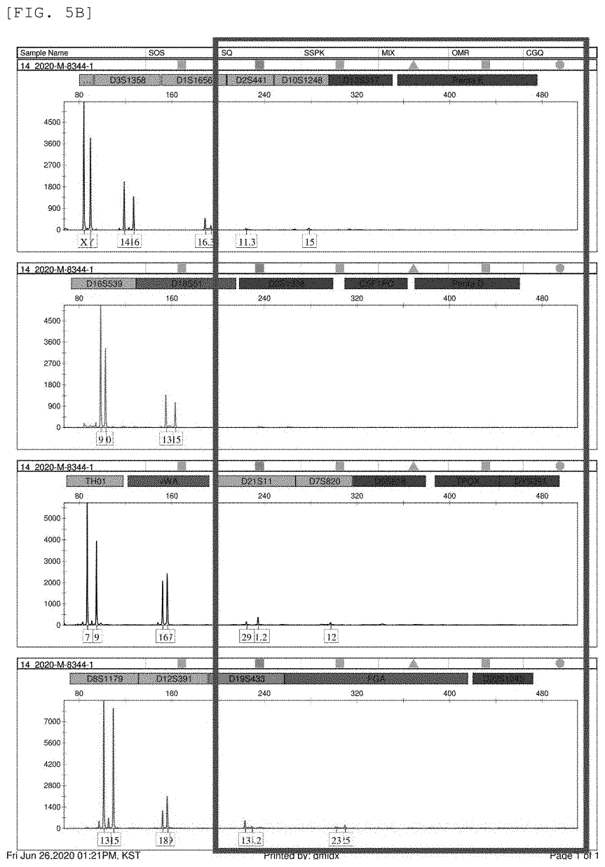

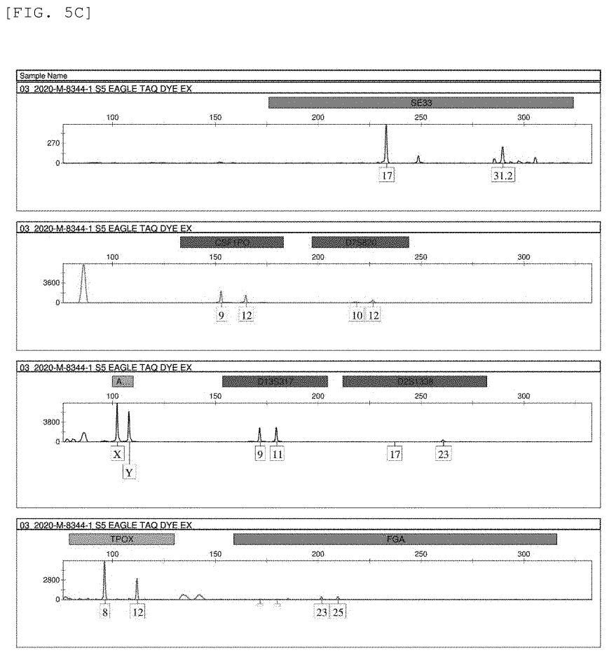

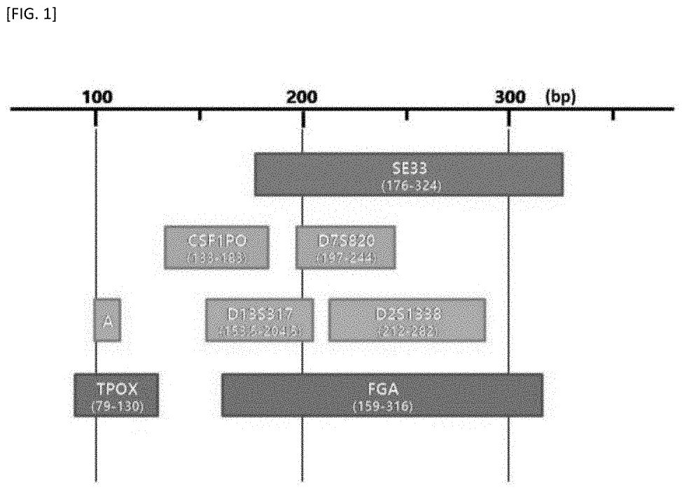

The mini-STR multiplex PCT kit including the primer set described above may apply the mini-STR technique that amplifies PCR amplification products while decreasing a size thereof in an STR region at each of the above 7 loci (SE33, CSF1P0, D7S820, D133317, D2S1338, TPOX and FGA). Further, the PCR kit of the present invention is configured to enable analysis of a mini-STR at SE33 locus and a mini-STR at TPOX, which could not be analyzed by AmpFLSTR™Minifiler™ PCT amplification kit (manufactured by ThermoFisher Scientific Co.) that is a mini-STR multiplex PCR kit commercially available for similar purposes, thereby improving gene identification sensitivity. When amplified by the multiplex PCR kit used for human genotype profile identification using a mini-STR combination of the present invention, a size distribution of multiplex PCT amplification products is as shown in FIG. 1.

TABLE 1
Primer set of mini-STR multiplex PCT Kit of the present
invention

| | | | | Expected |
|---|---|---|---|---|
| Sequence | S′ | Sequence information | | allele |
| number | marker | (5′→3′) | Loci | range |
| SEQ ID NO: 1 | VIC | Forward: | CSF1PO | 139-193 |
| | | TGCTAACCACCCTGTGTCTC | | bp (5-16) |
| SEQ ID NO: 2 | — | Reverse: | | |
| | | TCCTGTGTCAGACCCTGTTC | | |
| SEQ ID NO: 3 | VIC | Forward: | D7S820 | 202-246 |
| | | CGATTCCACATTTATCCTCATTG | | bp (5-16) |
| | | AC | | |
| SEQ ID NO: 4 | — | Reverse: | | |
| | | GGCTGACTATGGAGTTATTTTAA | | |
| | | GG | | |
| SEQ ID NO: 5 | PET | Forward: | TPOX | 81-129 bp |
| | | GAACAGGCACTTAGGGAACC | | (4-16) |
| SEQ ID NO: 6 | — | Reverse: | | |
| | | TAGGCCCTTCTGTCCTTGTC | | |
| SEQ ID NO: 7 | PET | Forward: | FGA | 160- 316 |
| | | AATGCCCCATAGGTTTTGAACT | | bp (12.2- |
| SEQ ID NO: 8 | — | Reverse: | | 51.2) |
| | | GATTTGTCTGTAATTGCCAGCA | | |
| SEQ ID NO: 9 | NED | Forward: | Amelogenin | 106-112 |
| | | CCCTGGGCTCTGTAAAGAATAG | | bp (XX and |
| SEQ ID NO: 10 | — | Reverse: | | XY) |
| | | ATCAGAGCTTAAACTGGGAAGC | | |
| SEQ ID NO: 11 | NED | Forward: | D2S1338 | 216-283 |
| | | AGAGGCCCTTGTCAGTGTTC | | bp (11-28) |
| SEQ ID NO: 12 | — | Reverse: | | |
| | | TTCCTACTGGCCCATAATCCA | | |
| SEQ ID NO: 13 | NED | Forward: | D13S317 | 162-210 |
| | | GTTCATTTCTTTAGTGGGATCC | | bp (5-17) |
| SEQ ID NO: 14 | — | Reverse: | | |
| | | AACTTGGGTTGAGCCATAGG | | |
| SEQ ID NO: 15 | 6-FAM | Forward: | SE33 | 184-330 |
| | | AGTGAAACTCCGTCAAAAGAAAG | | bp (3-39.2) |
| SEQ ID NO: 16 | — | Reverse: | | |
| | | CAACATCTCCCCTACCGCTA | | |

As described above, the primer set at the selected loci (excluding amelogenin locus) was desirably prepared for specific amplification of mini-STR regions targeted in the present invention. For this purpose, the primer sets in Table 1 were prepared such that a size of each amplified product in the mini-STR region becomes less than 330 bp with regard to the selected loci. The mini-STR multiplex PCR kit prepared a primer in which a size of the amplified product of the STR region is reduced by about 108 bp, as compared to the GlobalFiler™ PCR amplification kit. Likewise, the primer was prepared such that a size of the amplified product of the STR region is reduced by about 136 bp, as compared to the PowerPlex® fusion system.

Further, the mini-STR multiplex PCR kit of the present invention and the conventional STR kits such a PowerPlex® fusion system land GlobalFiler™ PCR amplification kit were compared to each other in terms of a size of amplified products of the STR region. The following Table 2 shows a reduction in size of the STR amplification product when the mini-STR multiplex PCR kit of the present invention (colors listed in Table 2 indicate a color of emitted light of fluorescent material labeled on the STR region amplified by a pair of primers to specifically amplify the STR region at each locus, wherein the fluorescent material is labeled at the 5′ end of a forward primer).

TABLE 2
Size comparison between commercially available STR
kits and amplified products

| | | Compared to | | Compared to | |
|---|---|---|---|---|---|
| | | PowerPlex ® | | GlobalFiler ™ | |
| | | fusion system | | PCR | |
| | | (reduction in | | amplification kit | |
| | | size of | | (reduction in | |
| | Allele | amplified | | size of | |
| Loci | range | product | | amplified product) | |
| CSF1PO | 133-183 bp | −178 | bp | −142 | bp |
| | (5-16) | | | | |
| D7S820 | 197-244 bp | −71 | bp | −60 | bp |
| | (5-16) | | | | |
| TPOX | 79-130 bp | −314 | bp | −255 | bp |
| | (4-16) | | | | |
| FGA | 159-316 bp | −97 | bp | −62 | bp |
| | (12.2-51.2) | | | | |
| D2S1338 | 212-282 bp | −12 | bp | −69 | bp |
| | (11-28) | | | | |
| D12S317 | 153.5-204.5 bp | −146 | bp | −43 | bp |

| | (5-17) | | | |
|---|---|---|---|---|
| SE33 | 176-324 bp | — | −128 | bp |

(3-39.2)

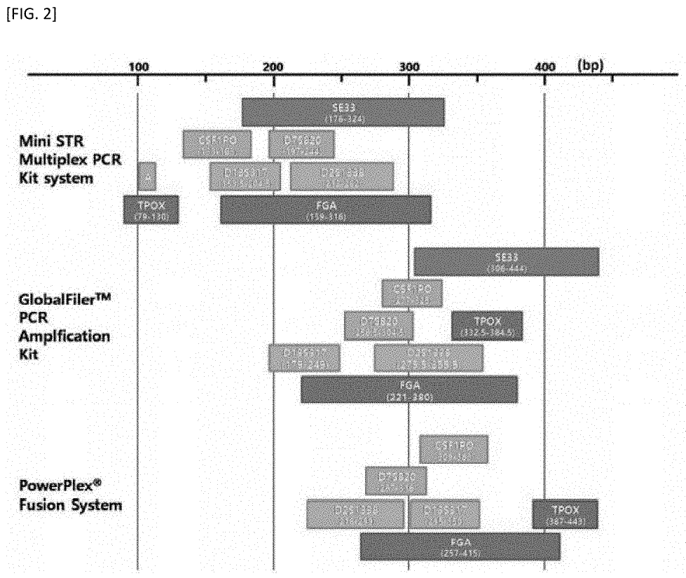

As shown in Table 2, when compared to PowerPlex® fusion system and GlobalFiler™ PCR amplification kit, the mini-STR multiplex PCT kit of the present invention adopted a mini-STR method that reduces a size of the STR amplification product by an average of 100 bp or more to design a primer set, thereby improving insufficient identification sensitivity of the above STR analysis kits. Results of comparing the sizes of STR amplification products between the mini-STR multiplex PCR kit of the present invention and the conventional kits, that is, the GlobalFiler™ PCR amplification kit and the PowerPlex® fusion system, are illustrated in FIG. 2.

Specifically, the mini-STR multiplex PCR kit of the present invention applied the mini-STR technique to the SE33 locus having high gene identification sensitivity, thereby improving humane genotype profile identification sensitivity. Further, it is possible to overcome a conventional problem of impossible cross-checking or cross-confirmation due to the SE33 locus which is included in analyzed loci of the GlobalFiler™ PCR amplification kit but is not included in analyzed loci of the PowerPlex® fusion system.

### Example 2: Preparation of Mini-STR Multiplex PCR Kit and STR Analysis Using the Same

The primer was prepared in a combined form of a multiplex primer set after determining whether specific amplification was present or not through a single amplification process using a DNA sample, that is, 2800M control DNA (produced by Promega Co.; 0.5 ng/μL, ×20 magnification diluted) as a template. In order to prepare a multiplex primer set, gradient PCR was performed to set an appropriate melting temperature (Tm). A fluorescent dye was labeled at the 5′ end of a forward primer specifically amplifying the STR region at each locus.

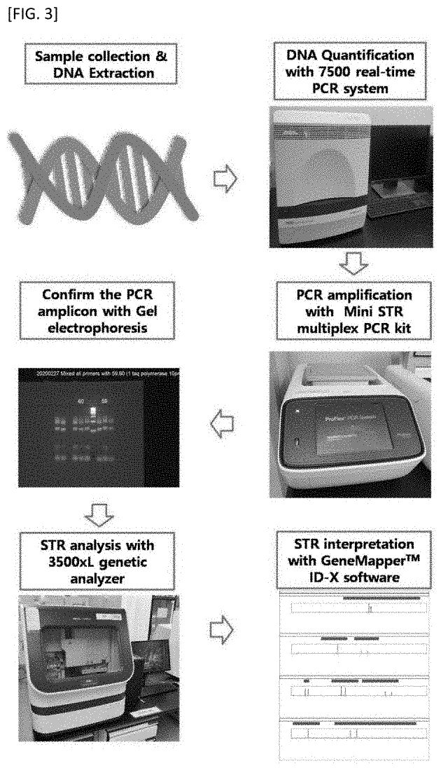

PCR amplification using the mini-STR multiplex PCR kit of the present invention was implemented using a ProFlex PCR system (manufactured by ThermoFisher Scientific Co.) with constitutional compositions in Tables 3 and 4 under PCR amplification conditions in Table 5. Further, the amplified amplicon was subjected to capillary electrophoresis, followed by final analysis using an analysis apparatus, that is, 3500×L genetic analyzer (manufactured by Applied Biosystems Co.). Further, GeneMapper™ ID-X software (produced by ThermoFisher Scientific Co.) was used for STR data analysis. As such, a process for human genotype profile identification (that is, an STR allele type analysis process) using the mini-STR multiplex PCR kit of the present invention is illustrated in FIG. 3.

TABLE 3
Components and contents of primer mix set

| Locus | Forward primer | Reverse primer |
|---|---|---|
| (primer sequence number) | (μM) | (μM) |
| CSF1PO (SEQ ID NOS: 1 and 2) | 0.2 | 0.2 |
| D7S820 (SEQ ID NOS: 3 and 4) | 0.2 | 0.2 |
| TPOX (SEQ ID NOS: 5 and 6) | 0.1 | 0.1 |
| FGA (SEQ ID NOS: 7 and 8) | 0.2 | 0.2 |
| Amelogenin (SEQ ID NOS: 9 and 10) | 0.15 | 0.15 |
| D2S1338 (SEQ ID NOS: 11 and 12) | 0.15 | 0.15 |
| D13S317 (SEQ ID NOS: 13 and 14) | 0.2 | 0.2 |
| SE33 (SEQ ID NOS: 15 and 16) | 0.4 | 0.4 |

TABLE 4
Multiplex PCR reaction composition in solution state

| Component | Volume (μL) |
|---|---|
| Primer mix set | 2 |
| 10× Gold STaR buffer (Promega) | 2 |
| Eagle Taq DNA polymerase (5 U/μL) | 1 |
| (Roche CUSTOMBIOTECH) | |
| Distilled water | 14 |
| DNA sample or control DNA | 1 |
| (2800M) 0.5 ng/μL) | |
| Total | 20 |

TABLE 5
Conditions of multiplex PCR reaction

| | Cycle | | | | | Number of |
|---|---|---|---|---|---|---|
| | step | Temperature | | Time | | cycles |
| Step 1 | Initial denaturation | 95° | C. | 11 | minutes | 1 |
| Step 2 | Denaturation | 95° | C. | 30 | seconds | 30 |
| | Annealing | 59° | C. | 30 | seconds | |
| | Extension | 72° | C. | 45 | seconds | |
| Step 3 | Final extension | 72° | C. | 15 | minutes | 1 |

| | | |
|---|---|---|
| 25° | C. | ∞ |

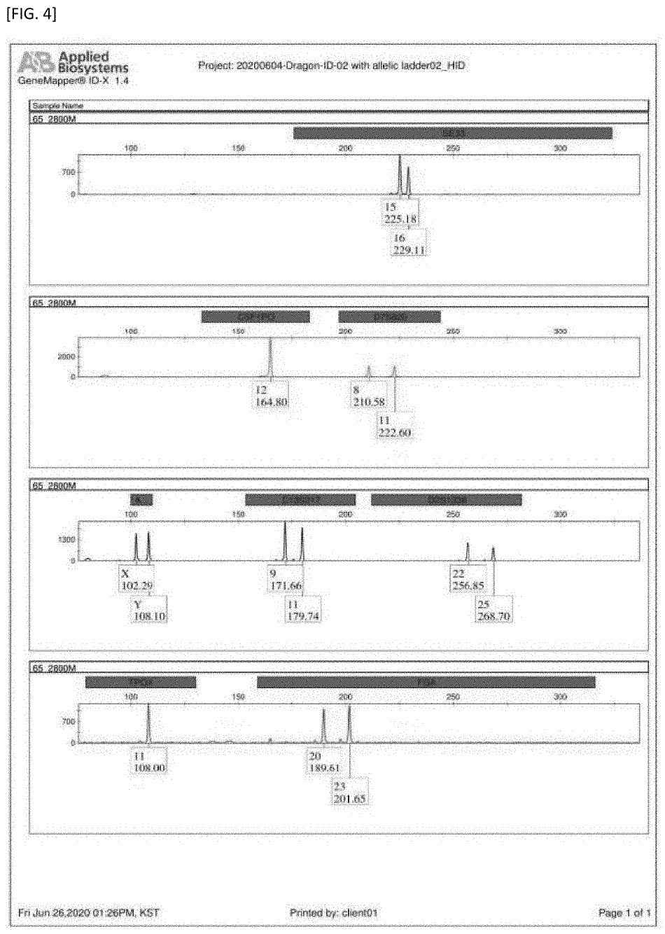

First, the obtained STR data value was compared to STR allele data value proposed for 280M control DNA of Promega Co., followed by confirming whether a mini-STR region at each locus is specifically amplified by the mini-STR multiplex PCR kit. In order to obtain a peak height balance between STR markers, a primer concentration ratio was adjusted and uniform multiplex STR data were obtained (FIG. 4). Accordingly, it could be seen that the mini-STR multiplex PCR kit of the present invention is suitable for human genotype profile identification through STR allele type analysis.

Next, the mini-STR multiplex PCR kit of the present invention was analyzed by comparing the kit to the conventional STR analysis kit. PCR composition and reaction conditions are as proposed in Tables 3, 4 and 5. For this purpose, 2800M control DNA of Promega Co. used as a standard DNA sample, K562 genome DNA, 9948 male DNA, control DNA 9947A of Applied Biosystems Co., and DNA control 007 were mixed and used as an allelic ladder. Further, DNA samples of 130 Koreans were used for comparative analysis experiments. In most of the samples, it was confirmed that the STR analysis result of the mini-STR multiplex PCR kit of the present invention coincides 100% with those of the GlobalFiler™ PCR amplification kit.

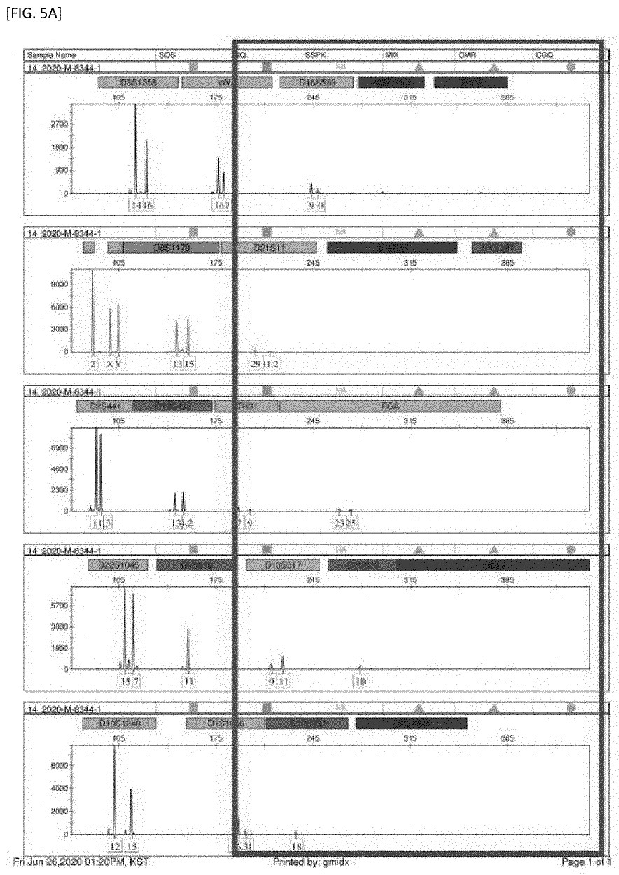

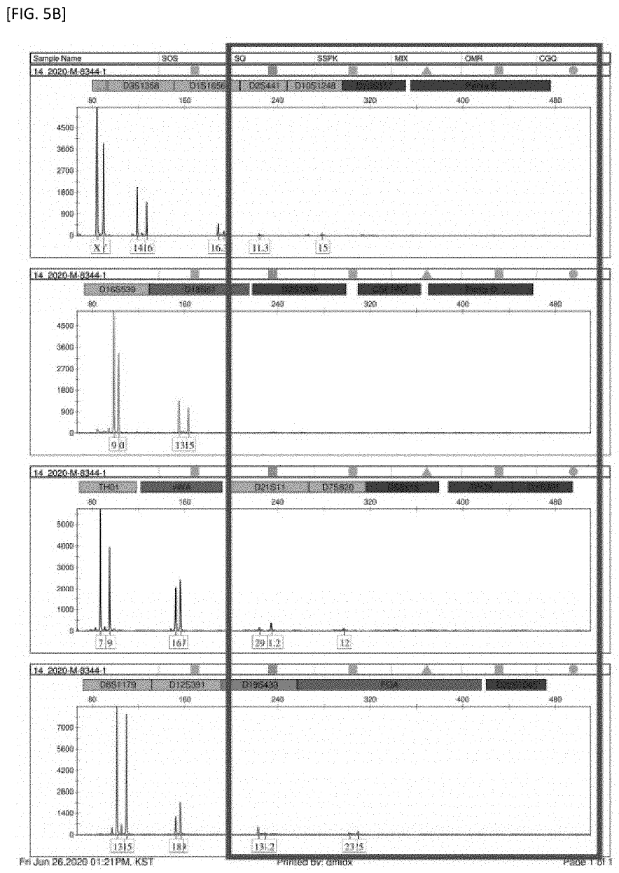

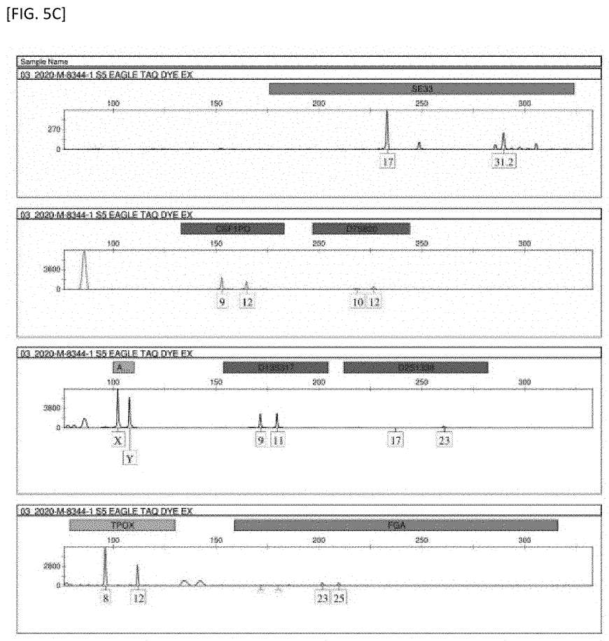

However, as a result of STR analysis of some DNA samples (samples inferred to entail denaturation or degradation of DNA) using the GlobalFiler™ PCR amplification kit and the PowerPlex® fusion system, STR allele types could not be discriminated at some loci (see FIGS. 5A and 5B). Results of five (5) loci (CSF1PO, TPOX, D7S820, SE33 and D2S1338) were not obtained from the GlobalFiler™ PCR amplification kit. Likewise, the PowerPlex® fusion system did not offer results of five (5) loci such as D13S317, D2S1338, CSF1PO, TPOX and D7S820.

On the other hand, as a result of analyzing sample DNA on scene, with which STR allele type analysis at some loci was failed by the GlobalFiler™ PCR amplification kit and the PowerPlex® fusion system, by means of the mini-STR multiplex PCR kit of the present invention, STR allele types could be discriminated at all of seven (7) loci (see FIG. 5c). Meanwhile, it was confirmed that these results of STR allele type analysis are substantially identical to those obtained using the GlobalFiler™ PCR amplification kit and the PowerPlex® fusion system through comparison of D13S317 and FGA loci. Accordingly, the mini-STR multiplex PCR kit of the present invention enabled STR analysis at the loci that failed to be analyzed by the GlobalFiler™ PCR amplification kit and the PowerPlex® fusion system (5 loci in each thereof). Further, STR analysis results at two (2) commonly analyzed loci were substantially identical to those obtained using the GlobalFiler™ PCR amplification kit and the PowerPlex® fusion system.

Based on the above experimental results, the mini-STR multiplex PCR kit of the present invention may identify mini-STRs at loci that could not be discriminated depending on DNA degradation level by the GlobalFiler™ PCR amplification kit and the PowerPlex® fusion system, which are commercially available in the art, so as to improve identification sensitivity which is insufficient in the prior art. Therefore, the mini-STR multiplex PCR kit of the present invention may identify STR at loci that could not be discriminated depending on DNA degradation level, thereby enhancing STR analysis success rate and offering a lot of DNA information. Further, the present invention may contribute to more efficient DNA identification such as extension of database record, increase in identity determination success rate, etc. Moreover, clues for reconstruction of a crime scene may be offered while improving reliability of forensic investigation results, simultaneously, thereby contributing to solving the crime.

As such, although the present invention has been described by way of the above examples, the present invention is not limited thereto. It will be understood that any alteration or modification may be possible by those skilled in the art without departing from the purpose and scope of the present invention, and such alteration and modification may also be included in the present invention.

1. A multiplex PCR kit used for human genotype profile identification (“gene identification”) using a mini-STR (short tandem repeat) combination, comprising:
a pair of primers of SEQ ID NOS: 1 and 2, which specifically amplifies STR region at CSF1P0 locus to produce a mini-STR amplification product at CSF1P0 locus;
a pair of primers of SEQ ID NOS: 3 and 4, which specifically amplifies STR region at D7S820 locus to produce a mini-STR amplification product at D7S820 locus;
a pair of primers of SEQ ID NOS: 5 and 6, which specifically amplifies STR region at TPOX locus to produce a mini-STR amplification product at TPOX locus;
a pair of primers of SEQ ID NOS: 7 and 8, which specifically amplifies STR region at FGA locus to produce a mini-STR amplification product at FGA locus;
a pair of primers of SEQ ID NOS: 11 and 12, which specifically amplifies STR region at D2S1338 locus to produce a mini-STR amplification product at D2S1338 locus;
a pair of primers of SEQ ID NOS: 13 and 14, which specifically amplifies STR region at D13S317 locus to produce a mini-STR amplification product at D17S317 locus; and
a pair of primers of SEQ ID NOS: 15 and 16, which specifically amplifies STR region at SE33 locus to produce a mini-STR amplification product at SE33 locus.

2. The multiplex PCR kit according to claim 1, further comprising a pair of primers of SEQ ID NOS: 9 and 10, which specifically amplifies amelogenin locus for sex discrimination.

3. The multiplex PCR kit according to claim 2, further comprising DNA polymerases, dNTPs, PCR buffer and a marker of PCR amplification product.

4. The multiplex PCR kit according to claim 3, wherein a first marker is labeled at both of 5′ end of the primer of SEQ ID NO: 1 and 5′ end of the primer of SEQ ID NO: 3, a second marker is labeled at both of 5′ end of the primer of SEQ ID NO: 5 and 5′ end of the primer of SEQ ID NO: 7, a third marker is labeled at 5′ end of the primer of SEQ ID NO: 9, 5′ end of the primer of SEQ ID NO: 11 and 5′ end of the primer of SEQ ID NO: 13, and a fourth marker is labeled at 5′ end of the primer of SEQ ID NO: 15.

5. The multiplex PCR kit according to claim 4, wherein the first marker, the second marker, the third marker and the fourth marker are fluorescent materials detected at different wavelengths, respectively.

6. The multiplex PCR kit according to claim 5, wherein mini-STR amplification products labeled with the fluorescent materials detected at different wavelengths, respectively, are discriminated through spectroscopic techniques, thereby simultaneously analyzing a plurality of STR allele types in only one examination.

7. A method for identification of a human genotype profile using a mini-STR combination, comprising:
preparing a gene sample from a specimen;
amplifying the gene sample through multiplex PCR using the multiplex PCR kit that is used for human genotype profile identification using a mini-STR combination as set forth in claim 1; and
determining STR allele types corresponding to CSF1P0 locus, D7S820 locus, TPOX locus, FGA locus, D2S1338 locus, D13S317 locus and SE33 locus, respectively, based on the result of the multiplex PCR.

8. The method according to claim 7, wherein a male or female gender is distinguished by XX type or XY type analysis of amelogenin locus.

9. The method according to claim 7, wherein the mini-STR amplification products labeled with the fluorescent materials detected at different wavelengths, respectively, are distinguished through spectroscopic techniques, thereby simultaneously analyzing a plurality of STR allele types by only one examination.


SEQUENCE LISTING (16 sequences)

SEQ ID NO: 1 - DNA, 20 bp, Artificial Sequence
  CSF1PO forward primer
tgctaaccaccctgtgtctc

SEQ ID NO: 2 - DNA, 20 bp, Artificial Sequence
  CSF1PO reverse primer
tcctgtgtcagaccctgttc

SEQ ID NO: 3 - DNA, 25 bp, Artificial Sequence
  D7S820 forward primer
cgattccacatttatcctcattgac

SEQ ID NO: 4 - DNA, 25 bp, Artificial Sequence
  D7S820 reverse primer
ggctgactatggagttattttaagg

SEQ ID NO: 5 - DNA, 20 bp, Artificial Sequence
  TPOX forward primer
gaacaggcacttagggaacc

SEQ ID NO: 6 - DNA, 20 bp, Artificial Sequence
  TPOX reverse primer
taggcccttctgtccttgtc

SEQ ID NO: 7 - DNA, 22 bp, Artificial Sequence
  FGA forward primer
aatgccccataggttttgaact

SEQ ID NO: 8 - DNA, 22 bp, Artificial Sequence
  FGA reverse primer
gatttgtctgtaattgccagca

SEQ ID NO: 9 - DNA, 22 bp, Artificial Sequence
  Amelogenin forward primer
ccctgggctctgtaaagaatag

SEQ ID NO: 10 - DNA, 22 bp, Artificial Sequence
  Amelogenin reverse primer
atcagagcttaaactgggaagc

SEQ ID NO: 11 - DNA, 20 bp, Artificial Sequence
  D2S1338 forward primer
agaggcccttgtcagtgttc

SEQ ID NO: 12 - DNA, 21 bp, Artificial Sequence
  D2S1338 reverse primer
ttcctactggcccataatcca

SEQ ID NO: 13 - DNA, 23 bp, Artificial Sequence
  D13S317 forward primer
gttcatttctttagtgggcatcc

SEQ ID NO: 14 - DNA, 20 bp, Artificial Sequence
  D13S317 reverse primer
aacttgggttgagccatagg

SEQ ID NO: 15 - DNA, 23 bp, Artificial Sequence
  SE33 forward primer
agtgaaactccgtcaaaagaaag

SEQ ID NO: 16 - DNA, 20 bp, Artificial Sequence
  SE33 reverse primer
caacatctcccctaccgcta